(19) United States
(12) Patent Application Publication
(10) Pub. No.: US 2022/0233359 A1
(43) Pub. Date: Jul. 28, 2022

(54) METHOD AND APPARATUS FOR RELIEVING HEADACHE SYMPTOMS

(71) Applicant: Zok Relief, LLC, Orem, UT (US)

(72) Inventors: John Hatch, Orem, UT (US); Spencer Glasgow, Provo, UT (US); Landon Bryner, St. George, UT (US)

(21) Appl. No.: 17/157,912

(22) Filed: Jan. 25, 2021

(51) Int. Cl. A61F 11/08

(52) U.S. Cl. CPC A61F 11/08

(57) ABSTRACT

An apparatus and method for treating symptoms associated with headaches. The apparatus may comprise a bulb, tube, and ear adapter, wherein the tube connects the bulb, in a substantially airtight manner, to the ear adapter. The ear adapter has a passage for gas, e.g., air, to flow from the tube and into an ear. The bulb may be flexible so that the it can be squeezed. Symptoms associated with headaches may be treated by engaging the ear adapter with the ear and performing a series of squeezes and releases with the bulb.

### BACKGROUND OF THE INVENTION

Migraines, other severe headaches, and headaches or head discomfort in general have long plagued humans. Although some headache or migraine attacks or episodes may be relatively short and/or mild, some people suffer from debilitating migraines that are long, severe, painful, and/or frequent. Doctors, professionals, and others have worked for years to develop and identify treatments and/or remedies for migraines or other headaches, and are always seeking treatments and/or remedies that are cheaper, simpler, safer, more convenient, more accessible, or otherwise better or improved.

What is needed is an apparatus and/or method for treating migraines or other headaches that is more inexpensive, easier to use/apply, safer, and/or more effective.

### BRIEF DESCRIPTION OF THE INVENTION

An apparatus and method for treating symptoms associated with headaches are disclosed. The apparatus, which may be referred to as a Pressure Changing Device, may comprise a bulb, tube, and ear adapter. The tube may be a substantially airtight connection between the bulb and the ear adapter, and may provide a passage for gas such as air to pass between the bulb and the ear adapter. The ear adapter may be tapered and shaped to fit an ear.

A method for treating symptoms associated with headaches may comprise engaging the ear adapter with an ear so create a substantially airtight connection between the ear adapter and the ear, performing a series of squeezes and releases with the bulb, and disengaging the ear adapter from the ear.

### BRIEF DESCRIPTION OF THE DRAWINGS

FIG. 1 shows an elevated angle view of an exemplary Pressure Changing Device.

FIG. 2 shows a side view of an exemplary Pressure Changing Device.

FIG. 3 shows an exemplary method for treating symptoms associated with headaches using an exemplary Pressure Changing Device.

FIG. 4 shows an exemplary side profile view of an exemplary bulb having a flat bottom.

FIG. 5 shows a side profile cross section view of an exemplary bulb having a flat bottom.

FIG. 6 shows an exemplary side profile view of an exemplary bulb having a flat bottom.

FIG. 7 shows a side profile cross section view of an exemplary bulb having a flat bottom.

FIG. 8 shows an alternative exemplary embodiment of an ear adapter.

FIG. 9 shows a side view of an exemplary connector for connecting tube to bulb.

FIG. 10 shows a side profile view of a portion of an exemplary connector for connecting tube to bulb.

FIG. 11 shows a side profile view of a portion of an exemplary connector for connecting tube to bulb.

FIG. 12 shows a side profile view of a portion of an exemplary connector for connecting tube to bulb.

FIG. 13 shows a side profile view of a portion of an exemplary connector for connecting tube to bulb.

FIG. 14 shows a side profile view of a portion of an exemplary connector for connecting tube to bulb.

FIG. 15 shows an exemplary method for treating symptoms associated with headaches using an exemplary Pressure Changing Device.

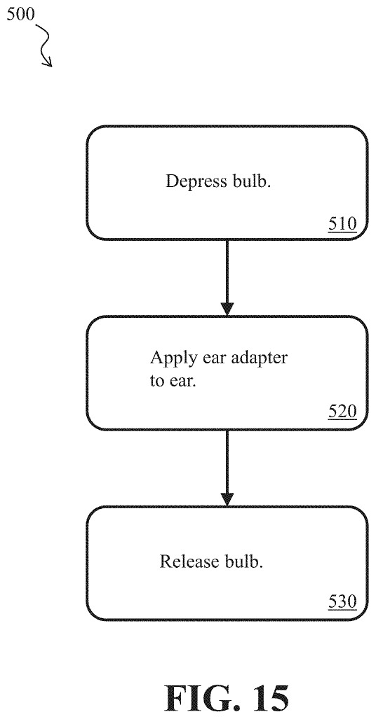

FIGS. 16a and 16b show how a connector may be inserted into bulb to create a seal.

FIG. 17 shows an exemplary connector for connecting tube to ear adapter.

### DETAILED DESCRIPTION OF THE INVENTION

This Application is a continuation-in-part of U.S. application Ser. No. 15/909,362, filed on Mar. 1, 2018 and titled “Method and Apparatus for Relieving Headache Symptoms,” the first inventor of which is John Hatch, and which is incorporated herein by reference in its entirety.

Application Ser. No. 15/909,362 claims priority to U.S. Provisional Application No. 62/466,248, titled “Method and Apparatus for Migraine Relief,” and filed on Mar. 2, 2017, and which is incorporated herein by reference in its entirety.

An improved method and apparatus are disclosed for treating migraines, pressure headaches, and other headaches.

In one embodiment, an apparatus is configured for pumping air and/or pressure into an ear, or for sucking (e.g., a vacuum effect) air and/or pressure out of an ear.

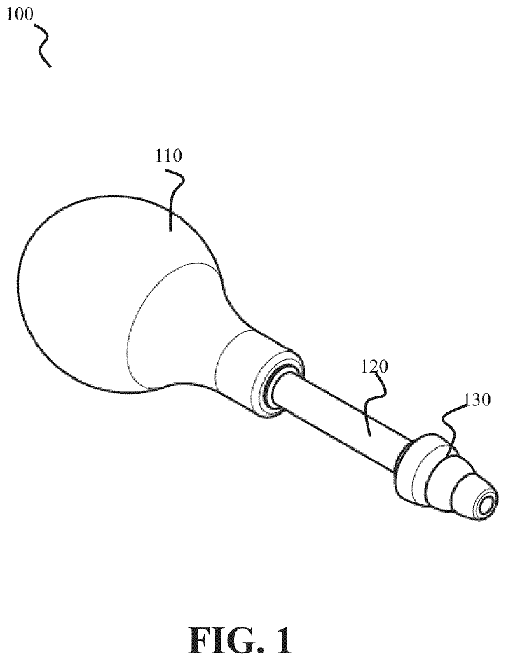

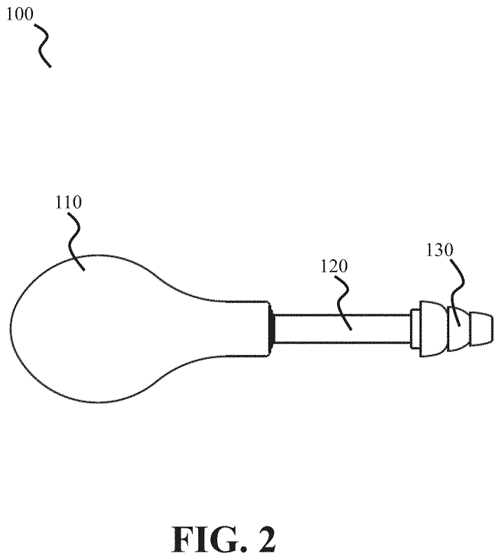

For example, one embodiment of the disclosed apparatus may be a Pressure Changing Device 100 as shown in FIGS. 1 and 2. As shown in FIGS. 1 and 2, Pressure Changing Device 100 may comprise bulb 110, hollow tube 120 and earpiece 130.

Bulb 110 may be any bulb-type apparatus or device known in the art for blowing air and/or creating pressure, or for sucking air and/or relieving pressure. Such bulbs are well known in, e.g., child nose mucus sucker devices, basting tubes for kitchen use, bulbs for inflating blood pressure checking equipment, and many other fields and/or uses. In one example, bulb 110 may be the bulb distributed with a Welch Allyn Insufflation Bulb for diagnostic otoscopes (Part number: 21504; GTIN 00732094086508). This type of bulb is shown in FIGS. 1 and 2.

The size of the Welch Allyn bulb 110 shown in FIGS. 1 and 2 is approximately 1.25 inches in diameter, with a length of approximately 2.25 inches.

Bulb 110 is only one solution for a source for producing air pressure or causing a vacuum result. Many machines and/or apparatuses are known in the art for creating, maintaining, and/or regulating a pressure differential between fluids such as air. For example, as may be known in the art, the tube could be connected and/or secured to a motorized air pump/vacuum which may provide air pressure or a vacuum. A motorized air pump/vacuum could be connected to and or controlled by a computer, microprocessor, electronic controller, manual on/off, increase, decrease, or other controls. For example, the pump/vacuum controller may include an interface through which a user may select a mode (either pump or vacuum or combination of both), a program (e.g., pump for three seconds, rest for three seconds, pump for three seconds, rest for three seconds; or vacuum for three seconds, rest for three seconds, vacuum for three seconds, rest for three seconds; or pump for three seconds; vacuum for three seconds; pump for three seconds; vacuum for three seconds), an air pressure or vacuum pressure, time settings, and other settings. The controller may thereby allow for customized treatments with the disclosed device.

As will be appreciated by one of skill in the art, many programs may be developed or devised based on length of pump or vacuum cycles, length of rest cycles, air or vacuum pressure, or any other variables associated with the disclosed apparatus.

In general, the disclosed apparatus may use or incorporate any device or solution known in the art, in one of many fields, that creates a pump or vacuum effect, and that may be able to regulate the strength and timing of such pump or vacuum effect. Such devices or solutions may be mechanical, manual, motorized, computer controlled, remotely controlled, battery operated, or have any other characteristics as may be known in the art.

An airtight tube 120 may be connected to the bulb 110. In general, this tube will be substantially flexible. Tube 120 may be made out of many materials or combinations of materials known in the art in the medical field and in many other fields involving flexible and airtight tubing. For example, tube 120 may be made out of plastic, rubber, fabric, or any other airtight tube. In general, tube 120 will be used at relatively low pressures and at room temperature, such that the tubing materials need not be designed or selected for extreme conditions and/or environments.

Tube 120 may be any color. In some embodiments, transparent, or translucent, or substantially transparent or substantially translucent tubing may be beneficial as it may allow for monitoring of cleanliness and/or possible blockage of the tube.

In general, tube 120 may be any length. In one embodiment, tube 120 may be three to 12 inches long. In general, if tube 120 is too short it may be inconvenient to use Pressure Changing Device 100 because a caretaker or user may have to hold the Pressure Changing Device 100 very close to his or her or the patient's ear, or may be restricted in locations in which the Pressure Changing Device 100 can be held or used, or in ways in which Pressure Changing Device 100 can be oriented during use. If tube 120 is too long, it may become tangled or unwieldy for use. Also, if tube 120 is too long, or if the volume of tube 120 is too large (tube volume is a function of tube length and tube diameter), the size of bulb 110 may need to increase so that bulb 110 is large enough to generate or relieve sufficient air pressure. In general, relative to the volume of tube 120, bulb 110 must be of a sufficient size to generate the desired air pressure or to generate the desired vacuum effect. A bulb 110 that is too small may not generate sufficient pressure or vacuum effect. A bulb 110 that is too large may be susceptible to generating too much pressure or vacuum, or a dangerous amount of pressure or vacuum.

In some embodiments, the length of tube 120 may be adjustable, or may be extendable by adding segments, or may be amenable to shortening by removing segments.

In one embodiment, the diameter of tube 120 may be 0.25 inches. In some embodiments, the diameter of tube 120 could vary over the length of tube 120.

In some embodiments, the cross section of tube 120 may not be circular, but may be another shape, e.g., square, or combination of shapes, or the shape may vary along the length of tube 120. In general, tube 120 may be any airtight apparatus with a passage for airflow.

The thickness of the wall of tube 120 may be constant or vary as is known in the art.

Tube 120 may be connected, via an airtight connection, to bulb 110. Many devices and approaches are well-known in the art, including in the medical field, for making an airtight connection with a tube.

For example, as illustrated in FIGS. 1 and 2, tube 120 may be connected to bulb 110 using a barbed connector. In some embodiments, the barbed connector may be a part of bulb 110 (monolithic), or may be connected to bulb 110. In other embodiments, the barbed end may be a monolithic part of tube 120, and may fit into a female bulb adapter. Barbed connections and fittings are well known in the art in the fields of medical devices, liquid and gas plumbing, and other fields.

In other embodiments, tube 120 may be glued to bulb 110, or welded to bulb 110, or heat welded to bulb 110, or monolithically manufactured as part of bulb 110, or secured with a threaded connection, or secured and/or attached in any other manner known in the art.

At the opposite end of tube 120, i.e., opposite the end to which bulb 110 is secured or located, is ear adapter 130.

In general, ear adapter 130 may be connected to tube 120 in the same manner as bulb 110 is connected to tube 120, e.g., using a barbed connection, or monolithically, or with any other tubing connection well known in the art as described herein relative to bulb 120.

In one embodiment, ear adapter 130 may be connected to tube 120 using a barbed connector that is part of tube 120.

In some embodiments, ear adapter 130 may be designed to be easily removable and/or replaceable, which may be important for cleanliness and/or sanitation reasons.

FIGS. 1 and 2 illustrate an exemplary ear adapter 130 connected and/or secured to the end of tube 120. FIGS. 1-2 illustrate a Welch Allyn diagnostic otoscope (Part number: 21504, GTIN 00732094086508), which is one exemplary embodiment.

In a preferred embodiment, ear adapter 130 is configured and/or designed to create an airtight seal with the ear for the purpose of pumping pressure into, or vacuuming pressure out of, the ear. In some embodiments, ear adapter 130 may be referred to as an earbud.

Ear adapter 130 has a canal or passageway through which air may travel from the tube, into and through the ear adapter, and into the ear, or be vacuumed out of the ear.

Ear adapter 130 may be manufactured out of plastic, foam, rubber, a combination and/or hybrid of such, of the material used in the Welch Allyn diagnostic otoscope (Part number: 21504, GTIN 00732094086508), or any other material, or combination or materials, known in the art.

In one embodiment, as shown in FIGS. 1-2, ear adapter 130 may be tapered to adapt to different sizes of ears and different variations of ear shapes and/or formations of ears. As shown in FIGS. 1 and 2, ear adapter 130 may have more than one tier. In some embodiments, one or more tiers of ear adapter 130 may be removed, e.g., cut off or sliced off, to adapt and/or customize an ear adapter to different patients and/or ear sizes, formations, or configurations.

Ear adapter 130 may be any color.

In general, ear adapter 130 may be any ear piece, disposable or non-disposable, known in many fields in the art for fitting in a human ear. Fields include medical, head phones for music and sounds, ear plugs, and other fields dealing with ears.

Ear adapter 130 may be manufactured in multiple sizes or configurations for human ears and configurations of varying sizes.

Ear adapter 130 may be designed to be configured for different sizes and configurations, e.g., with breakaway or removable parts or segments.

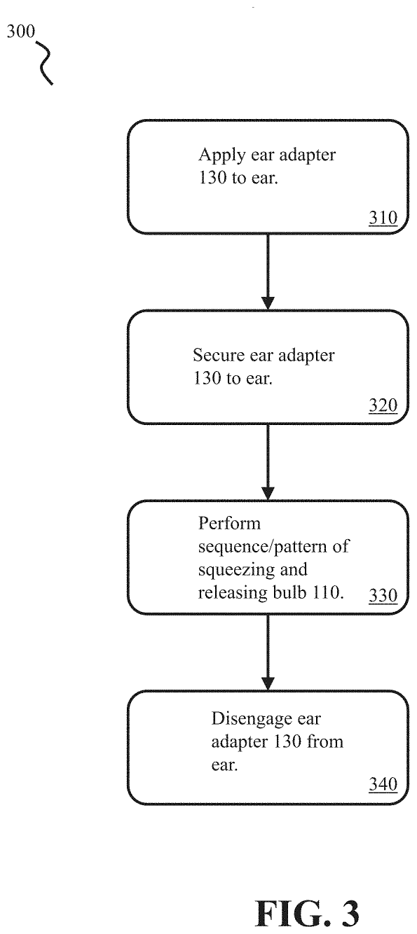

As shown in exemplary method 300 in FIG. 3, Pressure Changing Device 100 may be used or applied to treat or otherwise relieve headaches or symptoms associated with headaches. At step 310 ear adapter 130 may applied to the ear by inserting ear adapter 130 sufficiently to create an airtight seal between ear adapter 130 and the ear. At step 320, ear adapter 130 may be secured in place by manually holding it in place, or by using another device, e.g., a headband, to secure ear adapter 130 to the ear and to facilitate the air tight seal. In general, ear adapter 130 may remain in place in the ear without holding it manually or using another apparatus or device to hold ear adapter 130 in place. At step 330, with ear adapter 130 inserted into the ear, the user, or a different operator (e.g., a doctor or a parent), may perform a sequence or pattern of squeezing and releasing bulb 110, or by using another pressure/vacuum generating device attached to the end of tube 120. For example, the operator may squeeze bulb 110 to generate pressure, or may release bulb 110 to generate a vacuum effect. In general, the operator may operate Pressure Changing Device 100 apparatus manually, or by entering a program, or by selecting a program, or in any other manner described herein, according to any of the programs or approaches described herein. At step 340, when the pattern of squeezing and releasing has been completed, the user may disengage ear adapter 130 from the ear.

Many variations on application of Pressure Changing Device 100 may be applied depending on symptoms, diagnoses, patient characteristics, patient preferences, or any other factors known in the art. For example, in one embodiment, after inserting Pressure Changing Device 100 into a user's or patient's ear in a manner that creates an airtight seal, or a substantially airtight seal, bulb 110 may be repeatedly compressed, followed by removal of Pressure Changing Device 100 from the ear. In one example, bulb 110 may be compressed 20 times and then removed from the ear. This pattern may be repeated cyclically. For example, bulb 110 may be compressed 20 times and then removed from the ear for 20 minutes, and this may be repeated for multiple, e.g., five, iterations.

Another exemplary embodiment or application may comprise the following steps: (1) deflate bulb 110, (2) insert bulb 110 into ear, (3) release bulb 110 and hold in the ear for 20 seconds, (4) pull bulb 110 out of ear, (5) wait five seconds, and (6) repeat steps (1)-(5) up to five times. Steps (1)-(6) may be performed on each ear, once every three hours, up to eight times per day.

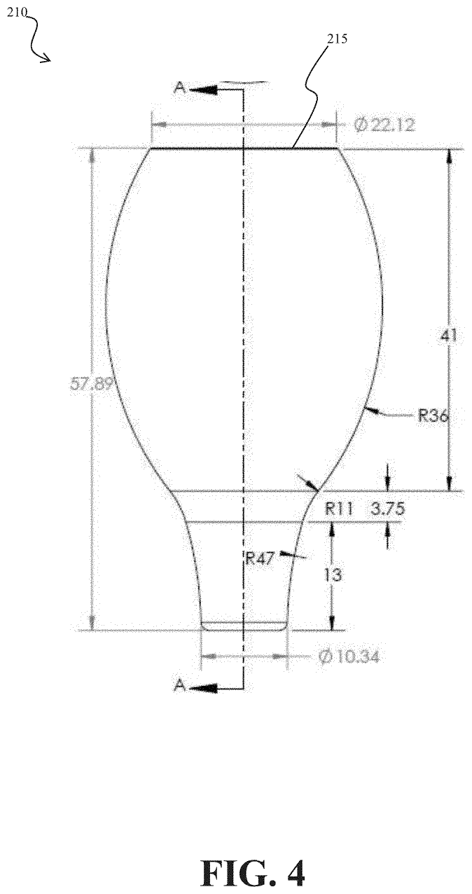

In one alternative embodiment, as shown in FIGS. 4-7, the bulb may have a flat bottom. FIG. 4 shows a side view of an exemplary bulb 210 with a flat bottom 215. FIG. 4 additionally shows exemplary dimensions for an exemplary bulb 210 with a flat bottom 215. In general, the plane of flat bottom 215 is perpendicular to the length of bulb 210. The diameter of flat bottom 215 may be approximately 22.12 mm. The diameter of flat bottom 215 may have many different lengths. The length of the diameter of flat bottom 215 may vary, and may depend on the size and dimensions of bulb 210, the desired finger engagement location on bulb 210, the desired air/fluid displacement properties of bulb 210, usability and convenience of a user to grip and engage bulb 210 with his/her fingers, and any other factor that may affect usability or aesthetics of bulb 210.

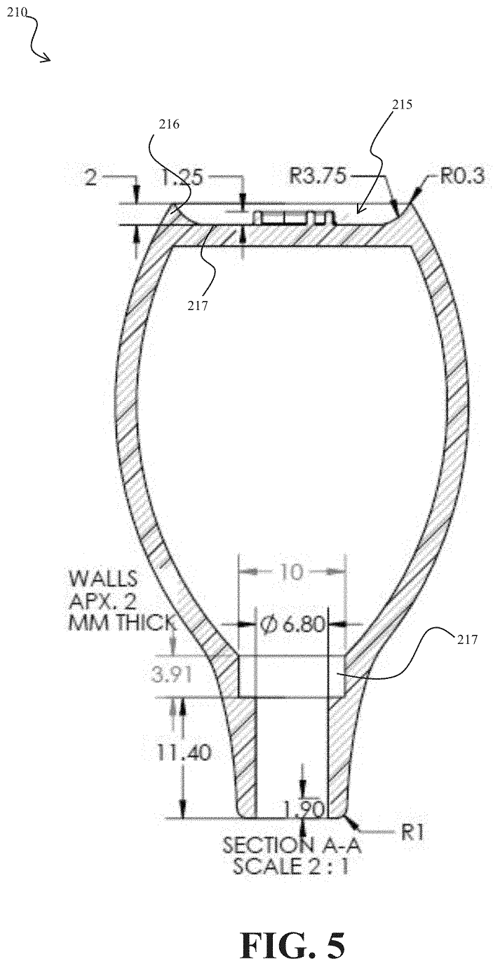

FIG. 5 shows a cross section of bulb 210. As shown in FIG. 5, flat bottom 215 may comprise a recessed crater 217 surrounded by a ridge 216. Although a flat bottom, on which bulb 210 may stand by itself, may be configured in many ways, using a ridge and crater system avoids instability that may result from imperfections in a completely flat bottom.

A flat bottom has several advantages. One advantage is that a flat bottom allows a device 200 (similar to device 100—but with a flat bottom) to stand upright, with the bulb on the bottom and the ear adapter pointing upward. Standing upright prevents device 200 from rolling, keeps the ear adapter from coming in contact with potential dirty surfaces, and allows for easy pickup by a user.

Additionally, adding a flat bottom allows for an easy way to alter the volume of the bulb. For example, by varying the location of the flat bottom along the length of the bulb, the volume of the bulb may be altered without changing the shape, curvature, or other dimensions of the bulb.

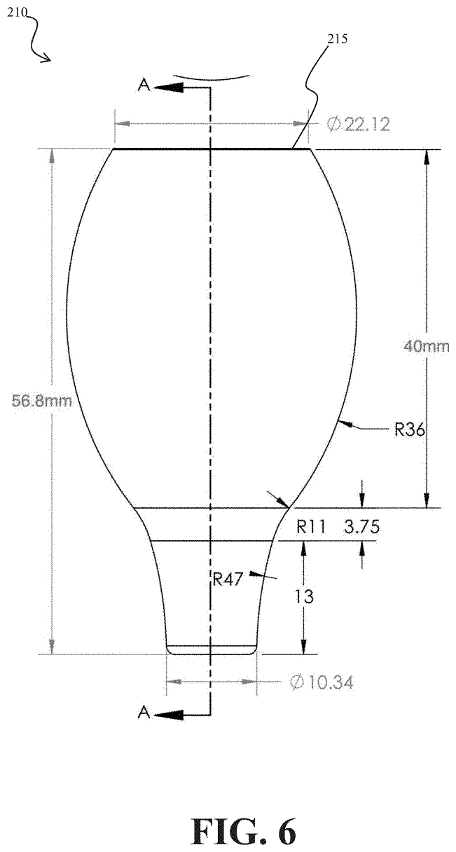

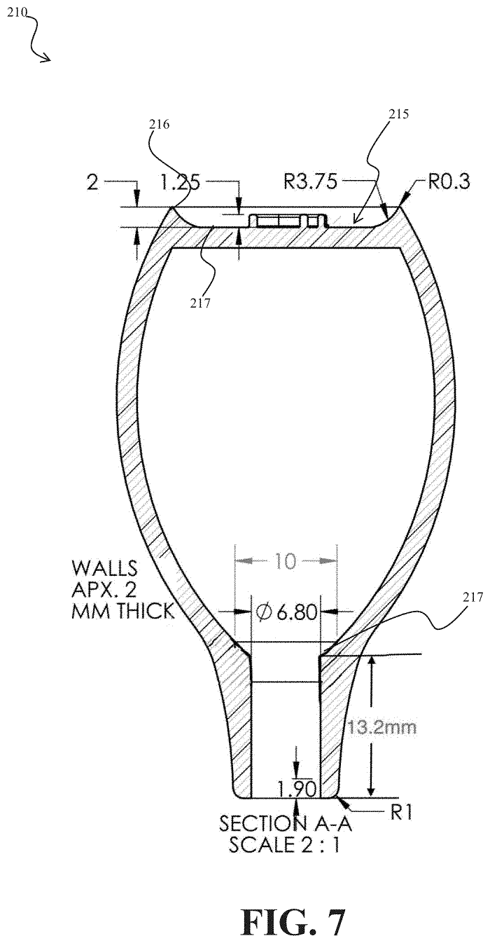

FIGS. 6 and 7 show a side view and a cross section of bulb 210 having slightly different dimensions from the dimensions in FIGS. 4 and 5.

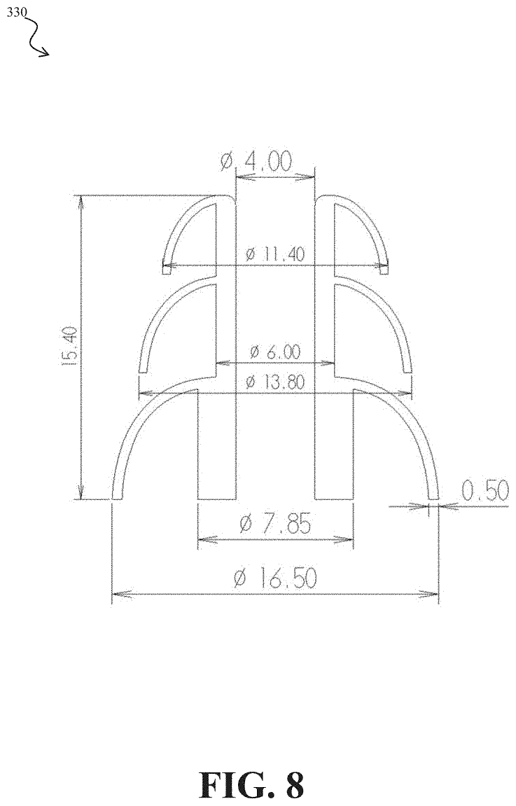

FIG. 8 shows an alternative exemplary embodiment of an ear adapter 330. FIG. 8 shows dimensions for ear adapter 330 in millimeters. Ear adapter 330 may be used just as ear adapter 130 is used and shown in FIGS. 1 and 2. The ear adapter dimensions shown in FIG. 8 are large enough to fit human ears and to create an improved seal between the interior of device 100 and a human ear. Ear adapter 330 may have similar qualities, characteristics, and may comprise similar or the same materials, as ear adapter 130 as disclosed herein.

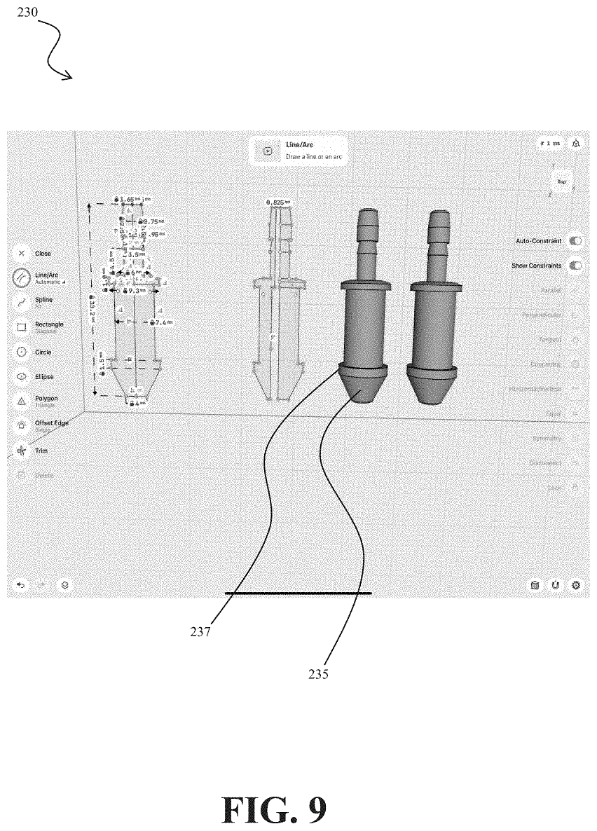

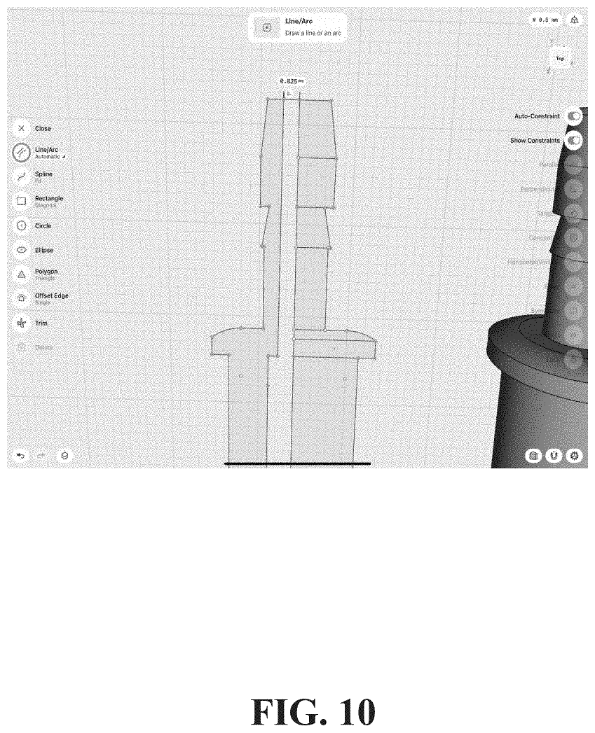

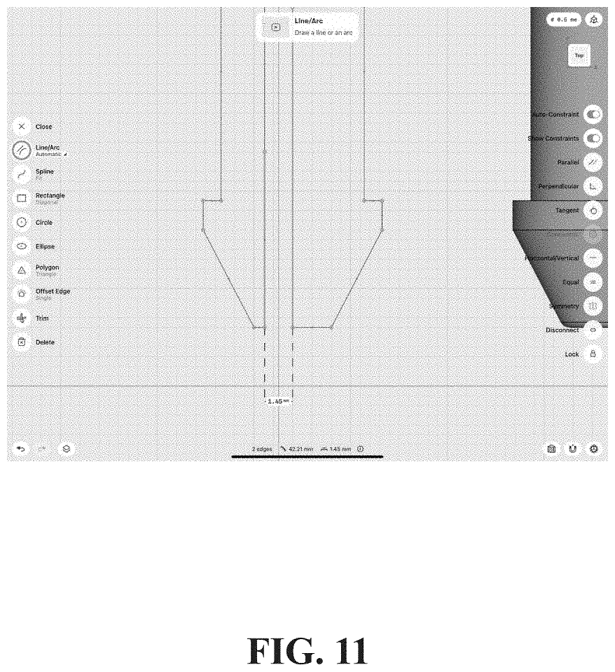

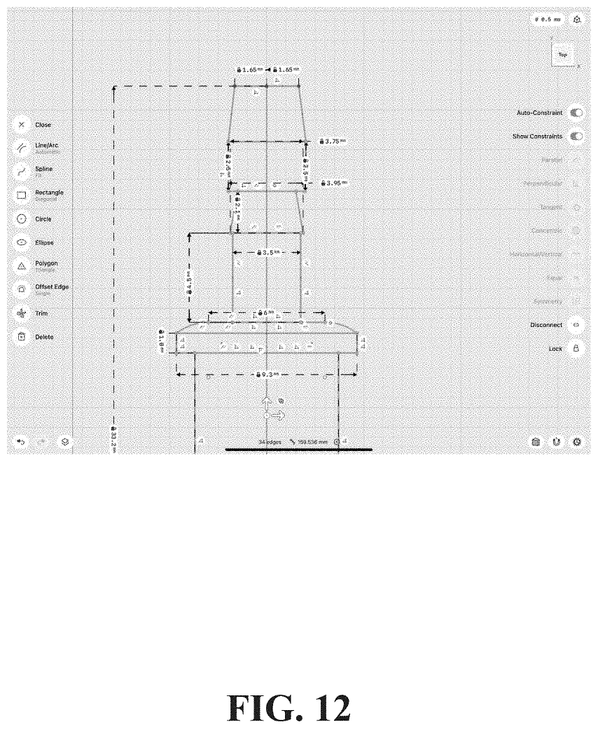

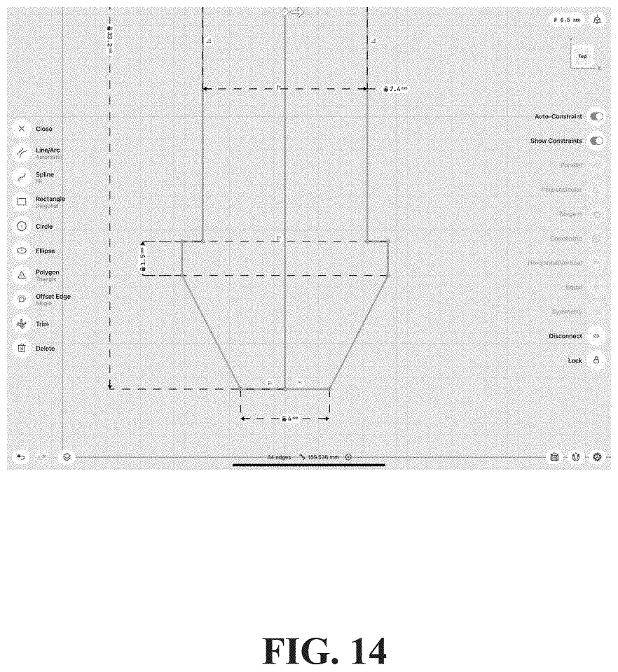

FIGS. 9-14 show various views of an exemplary connector for connecting tube 120 to bulb 110 or bulb 210. In one embodiment, as shown in FIGS. 5 and 7, bulb 210 may include interior flange/lip 217, which is dimensioned to complement tip 235 and flange 237 in connector 230.

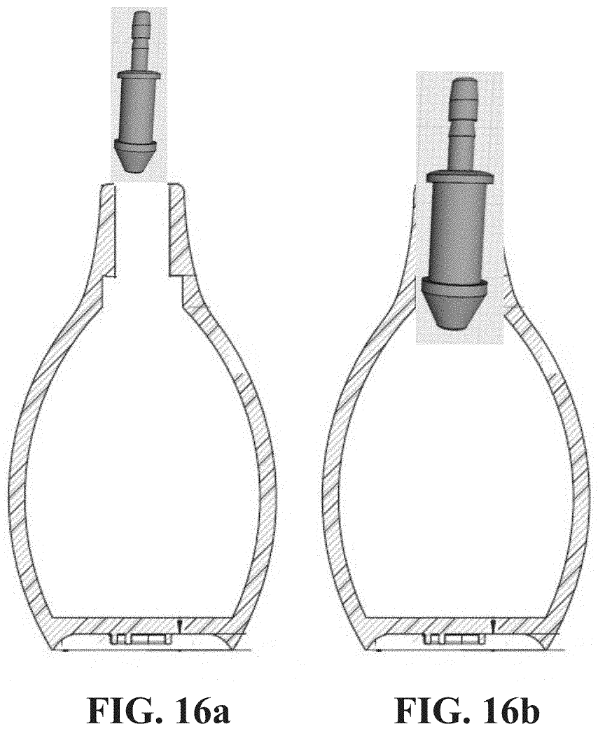

FIGS. 16a and 16b show how a connector may be inserted into bulb to create a seal. In one embodiment, flange in connector and lip in bulb may be designed with tolerances so that, at a specific pressure, the seal fails, thereby providing a safety feature to prevent a user from using excessive air/fluid pressure. For example, the tolerances of connector and bulb may be adjusted so that the seal fails at a desired pressure. This may be accomplished by adjusting dimensions. Modest trial and error may be required.

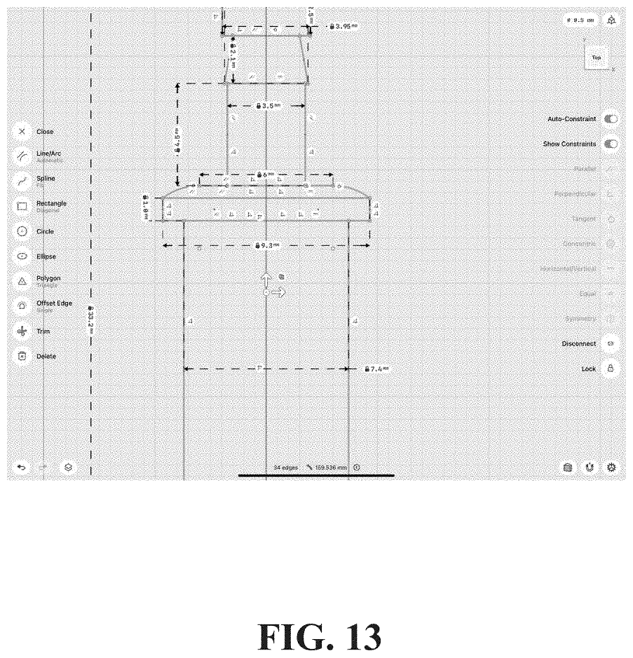

As shown in exemplary method 500 in FIG. 13, Pressure Changing Device 100 may be used or applied to treat or otherwise relieve headaches or symptoms associated with headaches. At step 510, the bulb is depressed. At step 520, the ear adapter is placed in a user's ear to engage the ear and ear canal. At step 530, the bulb may be released, thereby creating a vacuum effect.

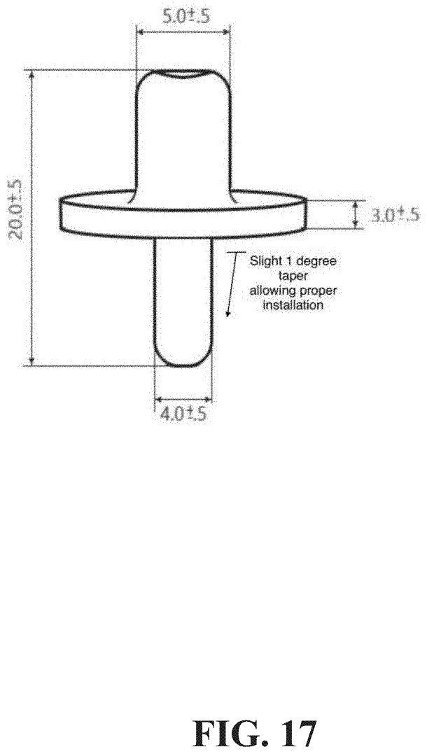

FIG. 17 shows an exemplary connector for connecting tube 120 to ear adapter 130.

It should be understood that the usage scenarios, patterns, and applications described herein are exemplary, and may be modified or altered for a variety of reasons, and remain within the scope of the disclosure herein. For example, the rate of inflation/deflation, frequency of inflation/deflation, rest periods between inflation/deflation, vigor/strength of inflation/deflation, and other characteristics of use of the disclosed apparatus may be varied for particular applications, desired outcomes, or circumstances.

1. A device for treating symptoms associated with headaches, comprising:
a pressure modifier comprising a bulb;
a connecting piece comprising a tube; and
an ear adapter;
wherein:
the bulb is configured to increase pressure and to release pressure;
the ear adapter has a channel for gas to flow into and out of the ear;
the ear adapter is configured to create a substantially airtight seal with an ear; and
the tube connects the bulb to the ear adapter and provides an airtight channel for gas to flow between the bulb and the ear adapter.

2. The device of claim 1, wherein:
the side of bulb opposite the tube is shaped so the device may stand on a flat surface with the tube pointed upward.

3. The device of claim 1, wherein the tube is secured to the bulb using a tube-bulb connector comprising an interior flange on the side of the tube-bulb connector that interfaces with the bulb.

4. The device of claim 1, wherein the tube is secured to the bulb using a tube-bulb connector, and the seal between the bulb and the tube-bulb connector is configured to fail at a particular pressure.

5. (canceled)

6. (canceled)

7. A method for treating headache symptoms, comprising:
engaging a pressure changing device with an ear; and
performing a pressure changing pattern with the pressure changing device;
wherein:
the pressure changing device comprises:
a pressure modifier comprising a bulb;
a connecting piece comprising a tube; and
an ear adapter; and
the ear adapter has a channel for gas to flow into and out of the ear;
the ear adapter is configured to create a substantially airtight seal with an ear; and
the tube connects the bulb to the ear adapter and provides an airtight channel for gas to flow between the bulb and the ear adapter.

8. The method of claim 7, wherein:
the side of bulb opposite the tube is shaped so the device may stand on a flat surface with the tube pointed upward.

9. The device of claim 7, wherein the tube is secured to the bulb using a tube-bulb connector comprising an interior flange on the side of the tube-bulb connector that interfaces with the bulb.

10. The device of claim 7, wherein the tube is secured to the bulb using a tube-bulb connector, and the seal between the bulb and the tube-bulb connector is configured to fail at a particular pressure.

11. (canceled)

12. (canceled)

13. (canceled)

14. (canceled)

15. (canceled)

16. (canceled)
